(19) United States
(12) Patent Application Publication
(10) Pub. No.: US 2024/0001054 A1
(43) Pub. Date: Jan. 4, 2024

(54) Mouthpiece For A Product Dispensing Device

(71) Applicant: Nemera La Verpillière, LA VERPILLIERE (FR)

(72) Inventors: Benjamin Quaglia, CHÂTEAUGAY (FR); Myriam Giraud, Belmont (FR); Laurent Vecellio None, CHAMBRAY LES TOURS (FR); Raphaël Vincey, Lyon (FR); Clement Dumet, BELLECOMBE EN BAUGES (FR); Thomas Leclercq, VILLEURBANNE (FR)

(73) Assignee: Nemera La Verpillière, LA VERPILLIERE (FR)

(21) Appl. No.: 18/038,147

(22) PCT Filed: Nov. 19, 2021
(86) PCT No.: PCT/EP2021/082372

§ 371 Date: May 22, 2023

(30) Foreign Application Priority Data
Nov. 20, 2020 (FR) 2011946

(51) Int. Cl. A61M 15/00

(52) U.S. Cl. CPC A61M 15/0021; A61M 15/0063; A61M 2202/064

(57) ABSTRACT

The invention relates to a nozzle for a device for dispensing product in powder form, having a dispensing duct, with a dispensing opening configured to open freely into an airway of a user, a grid arranged transversally across the dispensing duct, a solid wall arranged in the dispensing duct around the grid forming a dispensing restriction, the dispensing restriction having a flow section smaller than a minimum flow section of the dispensing duct. The invention also relates to a device for dispensing product in powder form by inhalation comprising such nozzle.

### FIELD OF THE INVENTION

The invention relates to the field of devices for dispensing product in powder form, in particular by inhalation.

### BACKGROUND OF THE INVENTION

The state of the art, in particular document EP1270034B1 already describes a dispensing device by inhalation of a product comprising a mouthpiece to dispense by inhalation a product in powder form, during an inspiration phase of a user, for example for a nasopharynx or pulmonary application. The product in dry powder form is contained initially in a capsule and is for example composed of an active product and lactose, to keep the product in powder form. The capsule is arranged in a housing, which communicates with the outside via holes, then a user perforates the capsule with needles, allowing the product in powder form to be released from the capsule. Once the capsule has been perforated, the user makes an inspiration. An air flow is thus created, which passes in the housing, around the capsule to move it and expel the powder. The air flow mixes with the powder generating the aerosol of medication. The aerosol then passes through the nozzle and is routed towards the user's airways. Thus, the product in powder form is dispensed by means of the air flow created by the user's inspiration. However, in some cases, the user's inspiration may not be very efficient, especially when the air flow is too weak.

When the air flow is too weak, the dry powder particles are not correctly deagglomerated. In fact, the air plays a role of deagglomerating or dispersing the powder particles, whether via the direct interaction of the air with the powder or indirectly via the rotation or the movement of the capsule containing the powder. When the particles are not correctly deagglomerated, they may be deposited in an unwanted area, for example the inner wall of the nozzle, the mouth or the throat, thereby reducing the effective amount of product inhaled.

Consequently, the dispensing quality provided by the device disclosed in this document largely depends on the inspiration quality, which means that it does not guarantee that the product in powder form is always dispensed efficiently.

To overcome this disadvantage, the invention aims in particular to propose a nozzle for a dispensing device of a product in powder form, which ensures that the product in powder form is dispensed more efficiently irrespective of the air flow generated.

### SUMMARY OF THE INVENTION

The invention therefore relates to a nozzle for a dispensing device of a product in powder form, comprising:
a dispensing duct, comprising a dispensing opening configured to open freely into an airway of a user,
a grid arranged transversally across the dispensing duct,
a solid wall arranged in the dispensing duct around the grid so as to form a dispensing restriction, the dispensing restriction having a flow section smaller than the minimum flow section of the dispensing duct.

This dispensing restriction thus corresponds to the smallest flow section of the entire dispensing duct, in other words to the smallest flow section of an air flow crossing the dispensing duct.

Thus, due to the dispensing restriction, in other words the restriction of the flow section of the air flow entraining the product, turbulences are generated in the air flow which combine with those generated by the holes in the grid and which improve the deagglomeration of the product. Consequently, the dispensing of product is improved. It is observed that by using the invention, there are fewer variations in the doses dispensed depending on the users. Such repeatability is particularly interesting since the inspirations may be quite different depending on the users, for example in the case of an athlete or a user suffering from respiratory difficulties. In addition, the invention thus proposes dispensing a more homogeneous quantity of product correctly deagglomerated and relatively independent of the inspiration quality, but also a dispensing that is more targeted towards the required area, in other words generally the lungs.

A transverse section or transverse arrangement designates a section or an arrangement along a plane arranged perpendicular to the longitudinal axis of the dispensing duct.

The words transverse, longitudinal, perpendicular, parallel, tangential or coplanar used in this document must be understood as including a certain tolerance, at least equal to the manufacturing variability, for example 10 degrees.

The nozzle may have the following characteristics, either alone or in combination:
The dispensing duct extends longitudinally;
The dispensing of product in powder form is carried out by inhalation;
The space extending longitudinally from the grid towards the dispensing opening is free, resulting in a dispensing duct that is particularly simple to manufacture and which is easy to demould;
The thickness of the grid is between 1 and 10 millimetres, preferably close to 1.3 millimetres or 2.5 millimetres;
The grid comprises a plurality of holes. Preferably, the grid comprises between 20 and 50 holes, more preferably between 35 and 40 holes;
The holes have a polygonal or circular shape, preferably a hexagonal shape;
The grid has a honeycomb structure. A honeycomb structure designates a series of holes of hexagonal section, in other words comprising 6 edges, wherein each edge of a hole is parallel to one edge of a different adjacent hole and wherein the distance separating each hole is constant;
The largest transverse dimension of the holes is less than or equal to the thickness of the grid;
The nozzle is a mouthpiece;
The grid is arranged at one end of the dispensing duct opposite to the dispensing opening. Thus, the product crossing the grid undergoes optimum deagglomeration before the product leaves via the dispensing opening;
The dispensing duct comprises a flow section that is constant or increasing from the grid up to the dispensing opening. Thus, less product is deposited in the dispensing duct, since its shape has no interior protrusions, thereby making dispensing of product more efficient;
The dispensing duct comprises a flow section of elliptical shape. Such a section is particularly adapted to the shape of the user's mouth;
The dispensing duct has a minimum inner dimension in transverse section which is between 15 and 30 millimetres, preferably between 20 and 25 millimetres, more preferably is equal to about 22.5 millimetres. Thus, we obtain a nozzle that has a relatively large inner diameter, such that the speed at which the product is transported is not too high, thereby making dispensing of product more efficient;
The dispensing duct is tubular and the minimum inner dimension in transverse section corresponds to its diameter;
The grid is circular;
The ratio S1/S2 between the area of the minimum flow section S1 of the dispensing duct and the area of the transverse section S2 of the grid is between 2 and 7. Thus, such a ratio can generate turbulences which improve the deagglomeration of the product;
The dispensing opening has a diameter D1, the grid has a diameter D2 and the ratio D1/D2 between the diameter D1 of the dispensing opening and the diameter D2 of the grid is between 1.1 and 5, preferably between 1.5 and 2.5, and more preferably equal to about 1.9. Thus, such a ratio can generate turbulences which improve the product deagglomeration and the dispensing repeatability;
“Diameter of the dispensing opening” designates the diameter of the smallest circle inscribed inside the opening in transverse section. It is understood that if the dispensing opening is circular, this is its diameter;
“Grid diameter” designates the diameter of the smallest circle surrounding the grid in transverse section, for example the circle circumscribed around the holes of the grid. It is understood that if the grid is circular, this is its outer diameter;
The surface of the solid wall oriented towards the dispensing opening is coplanar with the surface of the grid oriented towards the dispensing opening;

Since these surfaces are coplanar, there is a sudden increase in the flow section of the air flow at the grid outlet, which is different from the case, for example, where the solid wall would not be flat and would have a transverse section that increases or decreases progressively towards the dispensing opening, forming a funnel around the grid. The sudden discontinuity generated by the planar wall of the solid wall maximizes the generation of turbulences in the air flow, said turbulences combining with those generated by the holes in the grid since the grid and the solid wall lie in the same plane. This increase in the generation of turbulences further improves the deagglomeration of the product, therefore efficient dispensing of the product, irrespective of the air flow generated, in other words irrespective of the user's inspiration.
The grid is centered in a flow section of the dispensing duct. Centered means that the smallest circle surrounding the grid in transverse section and the smallest circle inscribed inside the dispensing duct in this transverse section are concentric. Thus, the dispensing of the product is improved;
The solid wall is made in one piece with the dispensing duct or with the grid or with the dispensing duct and the grid. Thus, manufacture is simplified, since there is one less assembly step;
The solid wall is assembled on the dispensing duct;
The nozzle is entirely made in one piece. Thus, manufacture is simplified, since there is no need for any assembly step to produce such a nozzle, in particular compared with a nozzle having an element oscillating in a chamber to generate turbulences;
The nozzle is made of a thermoplastic material, preferably polypropylene or acrylonitrile butadiene styrene;
The nozzle has a U-shape in longitudinal section, the web of the U being formed by the solid wall and the grid, and the branches of the U being formed by the dispensing duct.

The invention also relates to a dispensing device by inhalation of a product in powder form comprising a nozzle as described previously.

According to other optional characteristics of the dispensing device taken alone or in combination:
The dispensing device comprises a dosing chamber configured to contain product in powder form, said chamber comprising an air inlet and an outlet, the nozzle being connected directly to the outlet of the dosing chamber;
The dispensing device comprises means that can be actuated by a user and configured to break the integrity of a capsule containing product in powder form arranged in the dosing chamber;
The capsule is arranged transversally in the dosing chamber (not longitudinally).

### BRIEF DESCRIPTION OF THE DRAWINGS

The invention will be better understood on reading the following description, given solely by way of example and with reference to the accompanying drawings in which:

FIG. 1 is a perspective view of a dispensing device comprising a nozzle according to one embodiment.

FIG. 2 is a longitudinal cross-sectional view of the dispensing device shown on FIG. 1.

FIG. 3 is a top view of a part of the dispensing device shown on FIG. 1.

### DETAILED DESCRIPTION OF THE INVENTION

On all the figures, the same references refer to the same elements. The following embodiments are examples. Although the description refers to one or more embodiments, this does not necessarily mean that each reference concerns the same embodiment, or that the characteristics apply only to a single embodiment. Simple characteristics of different embodiments may also be combined to provide other embodiments.

The products, in particular pharmaceutical products, likely to be used in the dispensing device include for example formulations containing at least one active ingredient such as corticoids, glucocorticoids, peptides, proteins, hormones, active ingredients of biological origin, nucleotide-based active ingredients, such as for example DNAs, RNAs or oligonucleotides, active ingredients of molecular weight up to 1500 Da, polysaccharides, vaccines, enzymes, antibodies, antivirals, or nutritional formulations or a mixture thereof.

The products, in particular pharmaceutical products, likely to be used in the dispensing device can be used for the treatment and/or prevention of asthmas, chronic obstructive pulmonary diseases (COPD), cardiovascular diseases such as angina pectoris, heart attacks, influenzas A and influenzas B.

The products, in particular pharmaceutical products, likely to be used in the dispensing device are for example formulations containing at least one active substance such as salbutamol, fluticasone, salmeterol, budesonide, formoterol, tiotropium, beclomethasone, vilanterol, laninamivir, levosalbutamol, ipratropium, fenoterol, their salts, hydrates, solvates, stereoisomers and/or derivatives such as for example fluticasone propionate, tiotropium bromide, beclomethasone dipropionate, fluticasone fuorate, fluticasone propionate or ipratropium bromide. The formulations may also contain a combination of two or more active substances such as for example fluticasone with salmeterol, budesonide with formoterol, fluticasone fuorate with vilanterol, beclomethasone with levosalbutamol, fluticasone with formoterol, ipratropium bromide with salbutamol or ipratropium bromide with levosalbutamol.

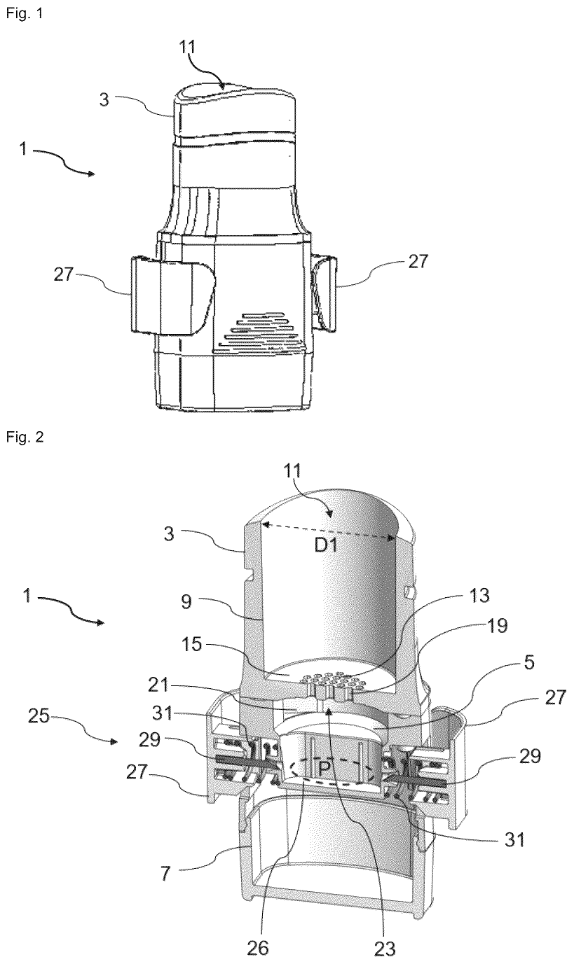

FIG. 1 shows, designated by the general reference 1, an inhalation dispensing device of a product in powder form.

As shown on FIGS. 1, 2 and 3, the dispensing device (1) of the product comprises a nozzle 3, a dosing chamber 5 and a storage chamber 7.

In this example, the nozzle 3 is a mouthpiece. It comprises a dispensing duct 9 extending longitudinally. The dispensing duct 9 comprises a dispensing opening 11 configured to open freely into an airway of a user, for example the mouth. The nozzle 3 also comprises a grid 13 arranged transversally across the dispensing duct 9. The nozzle 3 further comprises a solid wall 15 arranged in the dispensing duct 9 around the grid 13 so as to form a dispensing restriction 17, the dispensing restriction 17 having a flow section smaller than the minimum flow section of the dispensing duct 9. The space extending longitudinally from the grid 13 up to the dispensing opening 11 is free. In this example, the nozzle 3 is entirely made in one piece. The nozzle 3 is made of a thermoplastic material. As shown on FIG. 2, the nozzle 3 has a U-shape in longitudinal section, the web of the U being formed by the solid wall 15 and the grid 13, and the branches of the U being formed by the dispensing duct 9.

The dispensing duct 9 comprises a flow section that is constant or increasing from the grid 13 up to the dispensing opening 11. In this example, the dispensing duct 9 comprises a flow section that is constant from the grid 13 up to the dispensing opening 11. According to a variant not shown, the dispensing duct comprises a flow section of elliptical shape.

The dispensing duct 9 has a minimum inner dimension in transverse section which is between 15 and 30 millimetres, preferably between 20 and 25 millimetres, more preferably is equal to about 22.5 millimetres. More precisely in this example, the dispensing duct 9 is tubular and the minimum inner dimension in transverse section corresponds to its diameter D1.

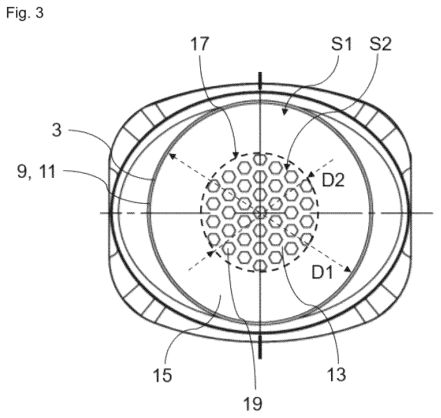

As shown on FIG. 2, the grid 13 is arranged at one end of the dispensing duct 9 opposite to the dispensing opening 11. The grid 13 is centred in a flow section of the dispensing duct 9. The thickness of the grid 13 is between 1 and 10 millimetres, preferably close to 1.3 millimetres or 2.5 millimetres. The grid also comprises a plurality of holes 19. Preferably, the grid 13 comprises between 20 and 50 holes, more preferably between 35 and 40 holes. In this example, the grid 13 comprises 37 holes. The holes 19 have a polygonal or circular shape, preferably a hexagonal shape. The grid 13 is circular and has a honeycomb structure, shown in particular on FIGS. 2 and 3. The largest transverse dimension of the holes 19 is in this example equal to 1.34 millimetres, less than or equal to the thickness of the grid which is 2.5 millimetres in this example. Thus, the holes 19 are formed as longitudinal holes. The ratio S1/S2 between the area of the minimum flow section S1 of the dispensing duct 9 and the area of the transverse section S2 of the grid 13 is between 2 and 7. The dispensing opening 11 has a diameter D1 equal to 22.5 millimetres, the grid has a diameter D2 equal to 11.6 millimetres and the ratio D1/D2 between the diameter D1 of the dispensing opening and the diameter D2 of the grid is equal to 1.9 in this example.

The solid wall 15 is made in one piece with the dispensing duct 9 and with the grid 13. As shown on FIG. 2, the surface of the solid wall 15 oriented towards the dispensing opening 11 is coplanar with the surface of the grid 13 oriented towards the dispensing opening 11. Such a nozzle 3 is for example obtained by injection moulding. Alternatively, according to a variant not shown, the solid wall is added to the dispensing duct, for example clipped in the dispensing duct.

The dosing chamber 5 is configured to contain product in powder form, said chamber comprising an air inlet 21 and an outlet 23, the nozzle 3 being connected directly to the outlet 23 of the dosing chamber 5. For example, the nozzle 3 is attached to the dosing chamber 5 by clipping. The air inlet 21 is arranged tangentially in the dosing chamber 5, the outlet 23 being arranged longitudinally on the side of the nozzle 3. Thus, an air flow entering via the air inlet 21 is swirling in the dosing chamber 5.

The dispensing device 1 further comprises means 25 that can be actuated by a user and configured to break the integrity of a capsule 26 containing product P in powder form arranged in the dosing chamber 5. The capsule 26 is arranged transversally in the dosing chamber 5. Thus, the capsule 26 is not arranged longitudinally in the dosing chamber 5. In this example, the means 25 are perforation means comprising two sliding buttons 27 arranged on each side of the dosing chamber 5, each supporting at least one needle 29 configured to perforate the capsule 26 and release the product P in powder form when an air flow passes in the dosing chamber 5. Each sliding button 27 comprises for example a return element, such as a spring 31. Thus, each return element is configured to return the respective sliding button 27 from a perforation position to a rest position.

Alternatively, according to a variant not shown, the perforation means are spikes.

The storage chamber 7 has the shape of a container. The storage chamber 7 is configured to store a plurality of capsules containing product in powder form. As shown on FIGS. 1 and 2, the storage chamber 7 is removably clipped to the dosing chamber 5.

In this example, it has been observed that the variability of the product dose dispensed efficiently depending on the air flow generated is much less compared with that of an existing commercial dispensing device of the above-mentioned type. Thus, for an air flow between 30 L/min and 100 L/min, the variability of the dose of product deagglomerated in the form of particles of size less than 5 μm and dispensed efficiently in the lungs of a user is approximately 15.6% whereas the commercial device has a variability of 28% under the same conditions.

The invention is not limited to the embodiments described and other embodiments will be clearly apparent to those skilled in the art. The nozzle 3 can have any outer shape, since this outer shape has no effect on the air flow passing inside the nozzle 3, in the dispensing duct 9.

### LIST OF REFERENCES

1: dispensing device
3: nozzle
5: dosing chamber
7: storage chamber
9: dispensing duct
11: dispensing opening
13: grid
15: solid wall
17: dispensing restriction
19: hole
21: air inlet
23: outlet
25: means
26: capsule
27: sliding button
29: needle
31: spring
D1: diameter of the dispensing opening 11
D2: diameter of the grid 13
S1: minimum flow section of the dispensing duct 9
S2: transverse section of the grid 13

1. A nozzle for a dispensing device for dispensing a product in powder form, comprising:
a dispensing duct having a minimum flow section and a dispensing opening configured to open freely into an airway of a user,
a grid arranged transversely across the dispensing duct,
a solid wall arranged in the dispensing duct around the grid forming a dispensing restriction, the dispensing restriction having a flow section smaller than the minimum flow section of the dispensing duct.

2. The nozzle according to claim 1, wherein the nozzle is configured as a mouthpiece.

3. The nozzle according to claim 1, wherein the grid is arranged at one end of the dispensing duct opposite the dispensing opening.

4. The nozzle according to claim 1, wherein the dispensing duct comprises a flow section that is constant or increasing from the grid up to the dispensing opening.

5. The nozzle according to claim 1, wherein the dispensing duct has a minimum inner dimension in a transverse section which is between 15 and 30 millimetres.

6. The nozzle according to claim 1, wherein a ratio between an area of the minimum flow section of the dispensing duct and an area of a transverse section of the grid is between 2 and 7.

7. The nozzle according to claim 1, wherein the dispensing opening has a diameter D1, the grid has a diameter D2 and a ratio D1/D2 between the diameter D1 of the dispensing opening and the diameter D2 of the grid is between 1.1 and 5.

8. The nozzle according to claim 1, wherein a surface of the solid wall oriented towards the dispensing opening is coplanar with a surface of the grid oriented towards the dispensing opening.

9. The nozzle according to claim 1, wherein the grid is centred in a flow section of the dispensing duct.

10. The nozzle according to claim 1, wherein the solid wall is made in one piece with the dispensing duct and/or with the grid.

11. The nozzle according to claim 10, wherein the entire nozzle is made in one piece.

12. A dispensing device for dispensing a product in powder form by inhalation, comprising the nozzle according to claim 1.

13. The dispensing device according to claim 12, further comprising a dosing chamber configured to contain the product in powder form, said dosing chamber comprising an air inlet and an outlet, the nozzle being connected directly to the outlet of the dosing chamber.

14. The nozzle according to claim 5, wherein the minimum inner dimension of the dispensing duct is between 20 and 25 millimetres.

15. The nozzle according to claim 7, wherein the ratio D1/D2 between the diameter D1 of the dispensing opening and the diameter D2 of the grid is between 1.5 and 2.5.
